(19) United States
(12) Patent Application Publication
(10) Pub. No.: US 2025/0209336 A1
(43) Pub. Date: Jun. 26, 2025

(54) METHODS AND DEVICES OF PROCESSING CYTOMETRIC DATA

(71) Applicants: AHEAD MEDICINE CORP, San Jose, CA (US); AHEAD INTELLIGENCE LTD., Taipei City (TW)

(72) Inventors: Chi-Chun Lee, Taipei City (TW); Bor-Sheng Ko, Taipei City (TW); Yu-Fen Wang, Taipei City (TW); Jeng-Lin Li, Taipei City (TW); Yun-Chun Lin, Taipei City (TW)

(73) Assignees: AHEAD MEDICINE CORP, San Jose, CA (US); AHEAD INTELLIGENCE LTD., Taipei City (TW)

(21) Appl. No.: 18/850,576

(22) PCT Filed: Mar. 29, 2023
(86) PCT No.: PCT/US2023/016649

§ 371 Date: Sep. 25, 2024

Related U.S. Application Data
(60) Provisional application No. 63/362,124, filed on Mar. 29, 2022.

(51) Int. Cl. G06N 3/09; G06F 17/16; G06N 3/045

(52) U.S. Cl. CPC G06N 3/09; G06F 17/16; G06N 3/045

(57) ABSTRACT

Disclosed are methods and devices of processing cytometric data. The present disclosure provides a method of processing cytometric data. The method comprises: dividing a first data matrix into a first plurality of first submatrices; encoding each of the first submatrices into one corresponding vector representation to acquire a first plurality of vector representations; and aggregating the first plurality of vector representations into a first ensemble representation. The first data matrix is indicative of a first plurality of properties of a first set of cells.

### CROSS-REFERENCE TO RELATED APPLICATIONS

This application claims a benefit and priority to U.S. Provisional Patent Application Ser. No. 63/362,124, filed on Mar. 29, 2022, which is hereby incorporated by reference in its entirety.

### FIELD OF THE INVENTION

The present disclosure relates to methods and devices of processing cytometric data. In particular, the present disclosure relates to methods and devices of processing cytometric data for hematologic malignancy classification.

### BACKGROUND

Hematologic malignancies are cancers of the blood, bone marrow, and lymph nodes. These hematologic malignancies are associated with substantial morbidity and mortality, and adversely affect quality of life. Hematologic malignancies must be precisely classified in order to select the appropriate therapeutic strategy for every new incoming patient. Hematologic malignancies arise from and, to varying degree, recapitulate the complex variety of cell lineages and stages of cell development that produce blood cells and other cells that respond to immunologic stimuli. This leads to distinct disease characteristics and prognoses. For example, acute lymphoblastic leukemia (ALL) and acute myeloid leukemia (AML) are caused by abnormality of lymphocytes and myelocytes, respectively. Acute promyelocytic leukemia (APL) is a subtype of AML, and other types of hematologic malignancies such as chronic lymphocytic leukemia (CLL) and a wide variety of lymphomas all have different symptoms, prognoses, and require different treatment strategies.

### SUMMARY OF THE INVENTION

Due to the wide variety of hematologic malignancies, a rapid and precise classification is critical to determine the first step towards effective disease management and cure for patients.

Flow cytometry (FC) generates data from high-throughput streams of individual cells from bone marrow, blood or lymphoid tissue specimens for high quality hematologic malignancy screening, diagnosis, and monitoring. Antibodies labeled with fluorescent tags permit characterization of the complex expression of cell proteins (i.e. antigens) by flow cytometry. Thousands or millions of cells in a specimen sample are evaluated using a panel of multiple antibodies, that are distinguished from each other by channels specific for the emission of the different fluorescent tags, producing large amounts of data points. Physicians or inspectors rely on visualization tools to display the fluorescence that indicates the expression of pairs of antigens among cell populations on two-dimensional scatter plots, and then they perform a hierarchical gating procedure to identify the abnormal cell populations. After the physicians or inspectors interpret multiple pairs of antibody-fluorescence combinations, the type of hematologic malignancy of a patient can then be determined in conjunction with the evaluation of morphologic findings and other appropriate testing. The manual gating process for FC is laborious and suffers from inter-physician subjectivity. Current tools assist the gating process and mostly only offer unsupervised cell population clustering. The purpose of these tools is to quickly identify the cell populations that seem to form related clusters; however, the ultimate procedure to generate an interpretation itself remains unchanged.

Differentiating types of hematologic malignancies is vital to determine therapeutic strategies for the newly diagnosed patients. Flow cytometry (FC) can be used as diagnostic indicator by measuring the multi-parameter fluorescent markers on thousands of antibody-bound cells, but the manual interpretation of large scale flow cytometry data has long been a time-consuming and complicated task for hematologists and laboratory professionals. Some embodiments lead to the development of representation learning algorithms to perform sample-level automatic classification. In this work, we propose a chunking-for-pooling strategy to include large-scale FC data into a supervised deep representation learning procedure for automatic hematologic malignancy classification. The use of discriminatively-trained representation learning strategy and the fixed-size chunking and pooling design are two of features of the framework provided in the present disclosure. It improves the discriminative power of the FC sample-level embedding (or pooling) and simultaneously addresses the robustness issue due to an inevitable use of down-sampling in conventional distribution based approaches for deriving FC representation. The framework provided in the present disclosure is evaluated on two datasets. Our framework outperformed other baseline methods and achieved 92.3% unweighted average recall (UAR) for four-class recognition on the UPMC (University of Pittsburgh Medical Center) dataset and 85.0% UAR for five-class recognition on the hema.to dataset. We further compared the robustness of our proposed framework with that of the traditional downsampling approach. Analysis of the effects of the chunk size and the error cases revealed further insights about different hematologic malignancy characteristics in the FC data.

The present disclosure provide novel methods or devices to efficiently process the cytometric data and thus can help physicians or inspectors efficiently to determine the type of hematologic malignancy of a patient. Additionally, the traditional methods of processing cytometric data cost too much computing power and memory space. The novel methods or devices provided in the present disclosure can save computing power, memory space, and the consumed electric power.

To deal with the bottleneck of the computing power and memory space, the operation of randomly downsampling the cytometric data is proposed. Such random downsampling may create unwanted variability but also run the risk of discarding important cells or residual tumor cells. The novel methods or devices provided in the present disclosure can reduce the unwanted variability and the risk of discarding important cells or residual tumor cells.

The exemplary embodiments disclosed herein are directed to solving the issues relating to one or more of the problems presented in the prior art, as well as providing additional features that will become readily apparent by reference to the following detailed description when taken in conjunction with the accompanied drawings. In accordance with various embodiments, exemplary systems, methods, devices and computer program products are disclosed herein. It is understood, however, that these embodiments are presented by way of example and not limitation, and it will be apparent to those of ordinary skill in the art who read the present disclosure that various modifications to the disclosed embodiments can be made while remaining within the scope of the invention.

An embodiment of the present disclosure provides a method of processing cytometric data. The method comprises: dividing a first data matrix into a first plurality of first submatrices; encoding each of the first submatrices into one corresponding vector representation to acquire a first plurality of vector representations; and aggregating the first plurality of vector representations into a first ensemble representation. The first data matrix is indicative of a first plurality of properties of a first set of cells.

Another embodiment of the present disclosure provides a device for processing cytometric data. The device comprises a processor and a memory coupled with the processor. The processor executes computer-readable instructions stored in the memory to perform operations. The operations comprise: receiving a first data matrix indicative of a first plurality of properties of a first set of cells; by means of the processor, dividing the first data matrix into a first plurality of first submatrices; by means of the processor, encoding each of the first submatrices into one corresponding vector representation to acquire a first plurality of vector representations; and by means of the processor, aggregating the first plurality of vector representations into a first ensemble representation.

### BRIEF DESCRIPTION OF THE DRAWINGS

In order to describe the manner in which advantages and features of the present disclosure can be obtained, a description of the present disclosure is rendered by reference to specific embodiments thereof, which are illustrated in the appended drawings. These drawings depict only example embodiments of the present disclosure and are not therefore to be considered limiting its scope.

FIG. 1 illustrates a schematic diagram showing a computer device according to some embodiments of the present disclosure.

FIG. 2 illustrates a schematic diagram showing operations of the framework according to some embodiments of the present disclosure.

FIG. 3 is a flowchart of a method according to some embodiments of the present disclosure.

FIGS. 4A to 4D illustrates confusion matrices showing the performance according to some embodiments of the present disclosure.

FIGS. 5A to 5D illustrates the performance and error distributions according to some embodiments of the present disclosure.

FIGS. 6A to 6D illustrates the results with different chunk quantity and chunk size according to some embodiments of the present disclosure.

FIGS. 7A and 7B illustrates the resulting distributions with the reduction of chunk quantity according to some embodiments of the present disclosure.

### DETAILED DESCRIPTION

The following disclosure provides many different embodiments, or examples, for implementing different features of the provided subject matter. Specific examples of operations, components, and arrangements are described below to simplify the present disclosure. These are, of course, merely examples and are not intended to be limiting. For example, a first operation performed before or after a second operation in the description may include embodiments in which the first and second operations are performed together, and may also include embodiments in which additional operations may be performed between the first and second operations. For example, the formation of a first feature over, on or in a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Time relative terms, such as “prior to,” “before,” “posterior to,” “after” and the like, may be used herein for ease of description to describe the relationship of one operation or feature to another operation(s) or feature(s) as illustrated in the figures. The time relative terms are intended to encompass different sequences of the operations depicted in the figures. Further, spatially relative terms, such as “beneath,” “below,” “lower,” “above,” “upper” and the like, may be used herein for ease of description to describe the relationship of one element or feature to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly. Relative terms for connections, such as “connect,” “connected,” “connection,” “couple,” “coupled,” “in communication,” and the like, may be used herein for ease of description to describe an operational connection, coupling, or linking one between two elements or features. The relative terms for connections are intended to encompass different connections, coupling, or linking of the devices or components. The devices or components may be directly or indirectly connected, coupled, or linked to one another through, for example, another set of components. The devices or components may be wired and/or wirelessly connected, coupled, or linked with each other.

As used herein, the singular terms “a,” “an,” and “the” may include plural referents unless the context clearly indicates otherwise. For example, reference to a device may include multiple devices unless the context clearly indicates otherwise. The terms “comprising” and “including” may indicate the existences of the described features, integers, steps, operations, elements, and/or components, but may not exclude the existences of combinations of one or more of the features, integers, steps, operations, elements, and/or components. The term “and/or” may include any or all combinations of one or more listed items.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

The nature and use of the embodiments are discussed in detail as follows. It should be appreciated, however, that the present disclosure provides many applicable inventive concepts that can be embodied in a wide variety of specific contexts. The specific embodiments discussed are merely illustrative of specific ways to embody and use the disclosure, without limiting the scope thereof.

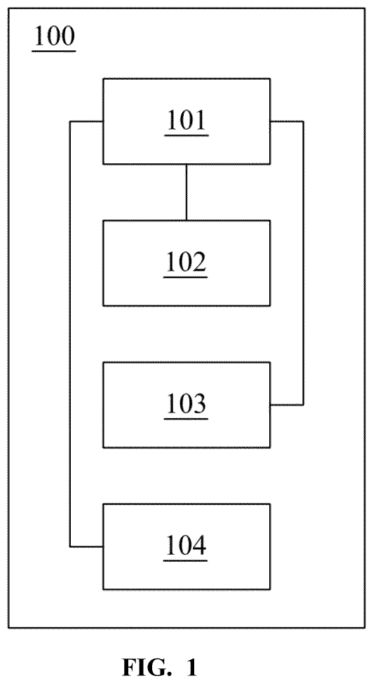

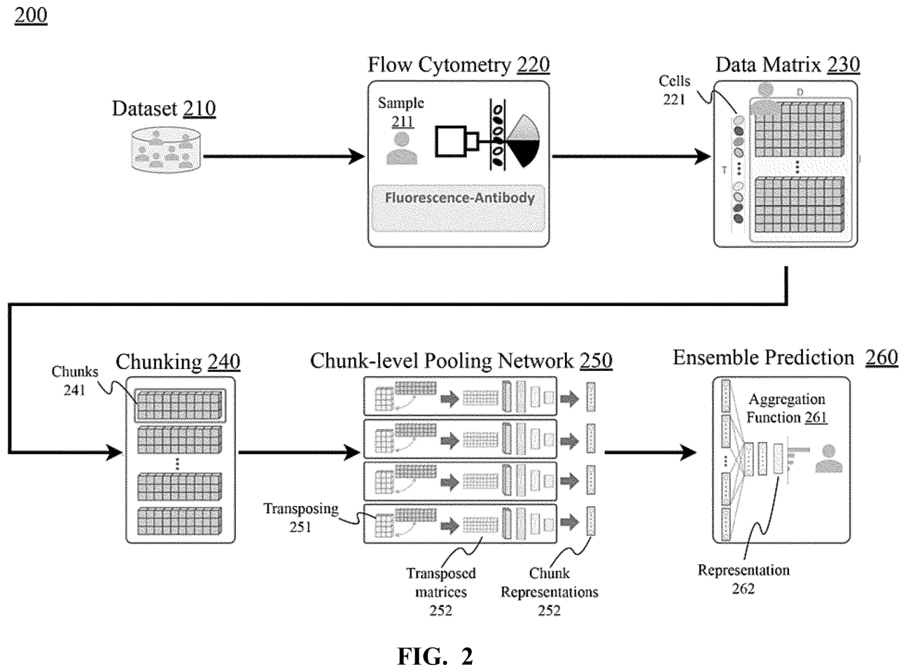

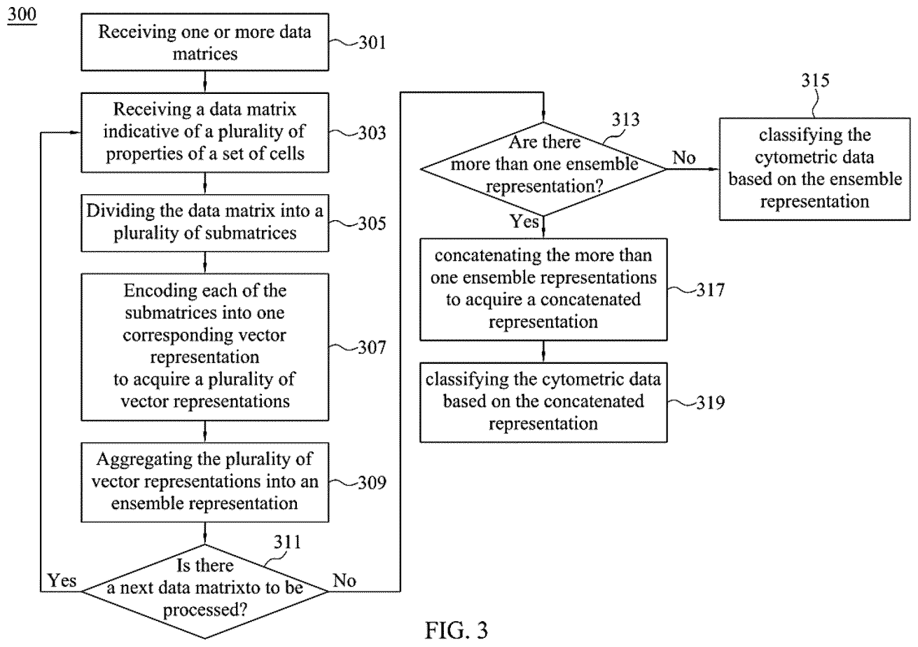

FIG. 1 illustrates a schematic diagram showing a computer device 100 according to some embodiments of the present disclosure. The computer device 100 may be capable of performing one or more procedures, operations, or methods of the present disclosure. The computer device 100 may be a server computer, a client computer, a personal computer (PC), a tablet PC, a set-top box (STB), a personal digital assistant (PDA), a cellular telephone, or a smartphone. The computing device 100 comprises processor 101, input/output interface 102, communication interface 103, and memory 104. The input/output interface 102 is coupled with the processor 101. The input/output interface 102 allows the user to manipulate the computing device 100 to perform the procedures, operations, or methods of the present disclosure (e.g., the procedures, operations, or methods disclosed in FIGS. 2 and 3).

The communication interface 103 is coupled with the processor 1101. The communication interface 103 allows the computing device 100 to communicate with data outside the computing device 100, for example, receiving data including cytometric data of a patient, the patient's information, the method, algorithm, program, or software to be performed, or the configurations of the method, algorithm, program, or software. The data received through the communication interface 103 may be stored in one or more databases outside the computing device 100.

A memory 104 may be a non-transitory computer readable storage medium. The memory 104 is coupled with the processor 101. The memory 104 has stored program instructions that can be executed by one or more processors (for example, the processor 101). Upon execution of the program instructions stored on the memory 104, the program instructions cause performance of the one or more procedures, operations, or methods disclosed in the present disclosure. For example, the program instructions may cause the computing device 100 to perform: receiving a first data matrix indicative of a first plurality of properties of a first set of cells; encoding the first brain image to generate a latent vector by the processor 101; dividing the first data matrix into a first plurality of first submatrices by the processor 101; encoding each of the first submatrices into one corresponding vector representation to acquire a first plurality of vector representations by the processor 101; and aggregating the first plurality of vector representations into a first ensemble representation by the processor 101. In some embodiments, the program instructions may cause the computing device 100 to perform: concatenating multiple ensemble representations to acquire a concatenated representation by the processor 101; and classifying the cytometric data based on the concatenated representation by the processor 101.

To alleviate these perennial issues of interpreting FC data, machine learning (ML) for FC data modeling are viable approaches. Most of these computational research work to identify cell-level characteristics to improve efficiency in the manual gating process. They build upon manual gating results for cell-based modeling. For efficient visualization, a handful of studies have utilized dimensionality reduction ML algorithms, e.g., Principal Component Analysis (PCA), t-Distributed Stochastic Neighbor Embedding (t-SNE), Uniform Manifold Approximation and Projection (UMAP), and Self-Organized Maps (SOM), to visualize FC data as scatter plots in the gating process. Other ML algorithms have been developed to automatically detect cell populations after cell clustering. For example, the recognition of blast cells was investigated. An autoencoder feature transformation approach has been widely used for detection of ALL minimal residual disease and rate cell population. Based on the detected cell types, the sample-level (specimen) outcome could only be derived by setting a pre-defined threshold to the total abnormal cell number.

Recently, classifying sample-level diagnostic outcomes has been found to possess more impact on clinical decision support than cell-level characterization. Hence, directly modeling sample-level FC data to perform disease type classification or disease status prediction is becoming the next step in automated modeling of FC data. Since the sample-level labels are obtained from the physician's or inspector's final interpretation, sample-level modeling can then be formulated as a supervised learning task without the dependency on the tedious effort of manual cell cluster labeling. However, in contrast to the aforementioned cell-level prediction that can easily represent each cell by a single vector composed of measured dimensions of multiple fluorescently labeled antibodies, adequately representing the entire FC data (i.e. the aggregate of fluorescent measurements for thousands or millions of cells) as a sample-level vector to be used in ML training is not as straightforward and is becoming a key technical endeavor.

In some embodiments, computing statistical functions is used as an intuitive method for vectorizing FC samples. One example is the representation of a sample as a percentage of over-threshold cells of each antibody to use as an input to classify subtypes of B-cell non-Hodgkin lymphomas. However, these statistics based approaches underestimate the data variability because the limited number of functions and empirically defined rules cannot fully capture the properties in the FC data sample.

Aside from representing the complex antigen expression by computing statistical functions, distributional approaches are becoming the state-of-the-art (SOTA) to encode the FC data as an embedding vector. The proportions of cells belonging to the learned Dirichlet-process Gaussian-mixture model (DPGMM) clusters may be computed as a representation. Other embodiments have used similar cluster-based feature distribution encoding approaches for ALL and AML MRD classification. Fisher Vector (FV) may be introduced, in which the gradients of clusters in Gaussian Mixture Model (GMM) is computed to obtain a sample-level representational vector, to distinguish AML and myelodysplastic syndromes (MDS) from normal samples. FV representations can be utilized to achieve SOTA accuracy for differentiating hematologic malignancy types. Even though the aforementioned distributional approaches are effective in learning sample-level FC representation, they remain discriminatively suboptimal. That is, these approaches treat the learning of sample-level representation and classifier training as two detached and independent modules. Instead of implementing sample-level representation learning and the outcome classifier at different stages, building a discriminative network in an end-to-end manner can better optimize the overall performances. Previous SOTA distributional modeling, i.e., FV encoding, consists of non-differentiable functions making it infeasible for end-to-end learning. We extended the FV encoding approach by introducing NetFV, which reorganized the latent assumption to enable discriminative gradient propagation in the deep layers for FC representation learning. The framework of NetFV considers the label back-propagation as conditions in learning the latent cell clusters beyond purely relying on unsupervised data clustering.

While these end-to-end approaches are appealing, the parameterized maximum likelihood optimization usually suffers from the GPU memory bottleneck during actual implementation. This technical issue becomes increasingly challenging and relevant because the need to detect different antigens is constantly evolving and the increasing capacity to simultaneously detect more and more fluorescent markers in a single tube. The growing diversity of hematologic malignancies that are recognized requires development of more customized marker panels, producing even higher dimensional FC data. Consequently, downsampling cells may be inevitable in some FC representation learning implementations. Random downsampling not only creates unwanted variability but also runs the risk of discarding important cells or residual tumor cells.

The present disclosure addresses two major issues in FC data modeling: suboptimal representations, and incomplete data usage, by developing a deep discriminative FC representation learning framework. One of the features of the present disclosure is a “chunking-for-pooling” process. The chunking-for-pooling process segments each FC data into fixed-sized “chunks,” pools cells into a chunk using a supervised network, and then aggregates the extracted chunk-level embedding vectors using an ensemble classification strategy. The GPU memory bottleneck issue can be solved due to the use of chunks, and the pooling mechanism is jointly optimized with the supervised network weights. In some embodiments of this framework, the chunk-level representation may be learned by a deep embedding network, such as NetFV and NetVLAD, which jointly optimizes the latent GMM assumption and the encoding parameters in an end-to-end discriminative manner. In the hematologic malignancy classification task, this embedding network embeds different diseases' discriminative information within each chunk, and maximizes the usage of FC data to its entirety (because no downsampling is applied).

The framework of the present disclosure is evaluated by using two hematologic malignancy datasets. The methods and devices using the framework of the present disclosure achieve a four-class 93.2% unweighted average recall (UAR) on the UPMC (University of Pittsburgh Medical Center) dataset and a five-class 85.0% UAR on the hema.to dataset. The chunking-for-pooling framework of the present disclosure was compared to the downsampling approaches to demonstrate robustness of the chunking-for-pooling framework. Further analyses of the prediction results and the effects of chunk size illustrated the properties of different malignancy categories and the details of the chunking-for-pooling framework. Here, we summarize the application scenarios in the major contributions of this paper listed as follows:
The present disclosure proposes a chunking-for-pooling framework that addresses the robustness issue in modeling FC data with varying numbers of cells across samples.
The present disclosure introduces deep embedding networks to learn discriminative sample-level FC representations.
The comprehensive experiments and analyses were conducted on two clinical datasets to validate the chunking-for-pooling framework of the present disclosure.

Sections A, B, and C describe two FC datasets and the chunking-for-pooling framework. Sections D, E, and F include experiments and results. Section G concludes the paper.

### A. DATASETS

### A.1. UPMC Dataset

The study was approved by the Institutional Review Board of University of Pittsburgh and the Research Ethics Committee of National Taiwan University Hospital. The present disclosure terms this dataset as the UPMC dataset. It contains bone marrow specimens collected at UPMC for patient care. There were 531 specimens from independent newly-diagnosed patients, for which we only enrolled FC data when a full five-tube panel for antibody-fluorescence measurements had been performed (shown in Table I). The antibody-fluorescence measurements may be optical measurements, such as forward and side light scatter. The diagnosis labels were derived from the comprehensive bone marrow evaluation done at UPMC, including morphologic evaluation, manual flow cytometry analysis, cytogenetic studies and other studies as needed (e.g. molecular studies). Table II and Table III show the class distribution of hematologic malignancies (APL, AML, ALL, and cytopenia) and the statistics of blast percentage, respectively.

Table I shows the fluorescence-antibody combinations used in the UPMC dataset and the hema.to dataset. The terms “FITC,” “PE,” “PerCP-Cy5-5,” “PE-Cy7,” “APC,” “APC-H7,” “V450,” “V500,” “KrOr,” “ECD,” “PC5.5,” “APCA750,” “PC7,” and “PacBlue” indicate the fluorophores to dye the cell to be inspected. The terms “Tube 1” to “Tube 5” indicate that the cells of one patient (e.g., one case) are divide into five tubes, and different antibodies would be inspected in different tubes. The terms “Tube 1” to “Tube 3” indicate that the cells of one patient (e.g., one case) are divide into three tubes, and different antibodies would be inspected in different tubes. One tube may be referred to as one sample in the present disclosure. The terms “CD36,” “CD15,” Kappa,” “CD16&57,” “FMC7,” “CD8,” “Lambda,” “IgM,” “HLA-DR,” and etc. indicate the antibody to be inspected.

TABLE I

| | | | PerCP- | | | | | |
|---|---|---|---|---|---|---|---|---|
| | | | Cy5- | PE- | | APC- | | |
| UPMC | FITC | PE | 5 | Cy7 | APC | H7 | V450 | V500 |
| Tube | CD36 | CD123 | CD64 | CD33 | CD34 | CD14 | HLA-DR | CD45 |
| 1 | | | | | | | | |
| Tube | CD15 | CD56 | CD117 | CD13 | CD11b | CD16 | HLA-DR | CD45 |
| 2 | | | | | | | | |
| Tube | Kappa | Lambda | CD5 | CDD19 | CD10 | CD38 | CD20 | CD45 |
| 3 | | | | | | | | |
| Tube | CD16&57 | CD7 | CD4 | CD3 | CD56 | CD8 | CD2 | CD45 |
| 4 | | | | | | | | |
| Tube | CD7 | CD13&33 | CD19 | — | CD56 | — | — | — |
| 5 | | | | | | | | |

| hema.to | FITC | PE | KrOr | ECD | PC5.5 | APC | APCA750 | PC7 | PacBlue |
|---|---|---|---|---|---|---|---|---|---|
| Tube | FMC7 | CD10 | CD45 | IgM | CD79b | CD23 | CD19 | CD20 | CD5 |
| 1 | | | | | | | | | |
| Tube | Kappa | Lambda | CD45 | CD38 | CD25 | CD103 | CD19 | CD11c | CD22 |
| 2 | | | | | | | | | |
| Tube | CD8 | CD4 | CD45 | CD3 | — | CD56 | CD19 | — | HLA- |
| 3 | | | | | | | | | DR |

Table II shows the data distribution of the two hematologic malignancy datasets. The term “Type” indicates different types of hematologic malignancy. The term “N” indicates the number of cases. The term “%” indicates the percentages of the numbers of cases of different types of hematologic malignancy to the number of total cases.

TABLE II

| UPMC | | | hema.to | | |
|---|---|---|---|---|---|
| Type | N | % | Type | N | % |
| AML | 200 | 37.66 | AML | 124 | 4.90 |
| APL | 32 | 6.03 | CLL | 305 | 12.06 |
| ALL | 131 | 24.67 | Lymphoma | 435 | 17.21 |
| Cytopenia | 168 | 31.64 | MM | 101 | 4.00 |
| Total | 531 | 100 | Normal | 1563 | 61.83 |
| | | | Total | 2528 | 100 |

Table III shows the blast percentage distribution of the UPMC dataset. The term “Type” indicates different types of hematologic malignancy. The terms “mean” and “SD” indicate the mean value and the standard deviation of the percentage distribution, respectively. The terms “Min” and “Max” indicate the minimum value and the maximum value of the percentage distribution, respectively. The terms “Q1,” “Q2,” and “Q3” indicate the first quartile, the second quartile, and the third quartile of the percentage distribution, respectively. The term “Q2” also indicates the median of the percentage distribution.

TABLE III

| Type | Mean | SD | Min | Q1 | Q2 | Q3 | Max |
|---|---|---|---|---|---|---|---|
| AML | 51.69 | 24.65 | 2.00 | 30.75 | 50.00 | 73.25 | 97.00 |
| APL | 76.81 | 19.94 | 8.00 | 75.00 | 84.00 | 88.00 | 96.00 |
| ALL | 74.25 | 21.43 | 3.50 | 64.98 | 83.00 | 90.00 | 99.00 |
| Cytopenia | 1.00 | 0.93 | 0.00 | 0.40 | 0.80 | 1.30 | 6.10 |

### A.2. Hema.to Dataset

The present disclosure uses another dataset collected in Munich Leukemia Laboratory (MLL) between Jan. 1, 2016 and Dec. 31, 2018 which was partially released for research. This dataset is termed as the hema.to dataset, based on the name of the technology demonstration website. The dataset comprises 20,622 routine diagnostic samples from patients with suspected B-cell neoplasms, of which 2528 samples are publicly available. As shown in Table II, we included the normal control, AML, and eight types of mature B-cell neoplasms, i.e., multiple myeloma (MM), chronic lymphocytic leukemia (CLL) and its precursor monoclonal B-cell lymphocytosis (MBL), prolymphocytic leukemia (PL) and hairy cell

leukemia (HCL), and four other B-cell lymphomas including marginal zone lymphoma (MZL), mantle cell lymphoma (MCL), follicular lymphoma (FL), and lymphoplasmacytic lymphoma (LPL). We regarded MBL, PL, HCL, and the four B-cell lymphomas as a single class called “lymphoma”. In the nine-color FC panel acquired on a Navios cytometer (Beckman Coulter, Miami, FL), three tubes had been run as shown in Table I along with the forward and side light scatter parameters, resulting in 26 unique fluorescence-antibody dimensions for FC data interpretations.

### B. DEEP CHUNKING-FOR-POOLING FRAMEWORK

FIG. 2 shows a schematic diagram of the framework 200 provided in the present disclosure. FIG. 2 discloses flow chart for one framework of the present disclosure. In the operation of chunking 240, flow cytometry data matrix 230 is split into chunks 241 (or submatrices). Each chunk 241 is fed into a chunk-level pooling network 250 to extract the corresponding chunk representation 252. In the chunk-level pooling network 250, the operation of transposing 251 may be performed on each of the chunks 241 (or submatrices). Finally, the ensemble prediction 260 is implemented by aggregating multiple chunk representations 252 by a function (e.g., an aggregating function 261) to predict the hematologic malignancy types.

The framework shown in FIG. 2 includes three parts. The first part refers to the preprocessing and chunking of FC data. The first part is described in Section B.1. The second part refers to the training approach and the architecture of the deep embedding networks used in the chunk-level pooling network 250. The second part is described in Section B.2. The third part refers to the ensemble classification method that aggregates the chunks to make a decision for each sample. The third is described in Section B.3.

### B.1. Chunk-Level Data Preprocessing

In FIG. 2, the dataset 210 may include the multiple samples of several patients. For the n-th (n E N) sample 211 (e.g., one tube of a patient) in the dataset 210, the FC dataXₙ ∈R^{T+D} can be acquired through fluorescence-antibody measurements performed by the flow cytometry 220. The FC data can be represented as a data matrix 220 having D columns and T rows. The FC data Xₙ ∈R^{T*D} were segmented, divided, or chunked into chunks of data X_{n,c} ∈R^{C*D}. The chunks of data may be represented chunks 241 or submatrices. Each of the chunks 241 or submatrices may have D columns and C rows. C is a constant chunk size. N is the total number of FC samples. D is the number of fluorescent-antibody combinations. T is the number of the cells in the FC data. Although the number of cells T are typically consistent across specimens, in real world situations, there are occasional variations and the results in a variable number of chunks

$${N}_{C} = \frac{T}{C} .$$

The number of chunks N_{c} is also called “chunk quantity” in the present disclosure to prevent confusion with the chunk size or other terms. There were an average of 8.82 chunks with 0.96 standard deviation per specimen in the FC data from the UPMC dataset and 14 chunks with 2.26 standard deviation in the hema.to dataset. Each chunk is assigned with the patient's hematologic malignancy category label for the chunk-level pooling network 250 (in which a deep embedding network is used) learning (described in Section B.2). The processes of chunking augment the data from the sample-level data by a factor of

$${N}_{C} = \frac{T}{C}$$

times in the supervised training. The augmented chunks will be aggregated by an ensemble mechanism or an aggregation function described in Section B.3.

### B.2 Deep Embedding Networks Used in Chunk-Level Pooling Network

In this stage, we aim to encode the cells in each chunk and represent the collective phenotype of the cells using latent network embedding. The present disclosure combines the immunophenotype representation learning and the classifier training in a discriminative deep network using a GMM probabilistic distribution assumption for the hematologic malignancy type classification tasks. In the traditional GMM-based encoding approaches, the GMM parameter λ includes the weight πₖ, mean μₖ, and covariance σₖ² for the k-th mixture to characterize mixtures of different cell types. The encoding approach calculates the gradients between each chunk-level FC data X_{n,c} simplified as X∈R^{C*D} to the GMM distribution learned from the entire training dataset. Therefore, the encoding vector can represent the relationship between that chunk to all the GMM mixtures based on a probability derived function. However, these representation learning approaches optimize the maximum likelihood in an unsupervised manner, which may not be optimal for the classification task. Some encoding functions can be rewritten by casting the parameters as learnable weights to enable end-to-end learning. As a result, the supervised deep network can optimize the latent GMM weights for the targeted hematologic malignancy labels. After training, we can then extract the latent embedding in the network (e.g., the chunk-level pooling network 250) to represent each chunk 241 of the sample 211.

Specifically, the present disclosure briefly describe the FC distributional encoding approach, Fisher Vector (FV), and another common GMM-based distributional encoding approach, Vector of Locally Aggregated Descriptors (VLAD). Then, we describe the key component that allows the gradient updating for FV and VLAD and therefore derive the corresponding end-to-end forms, NetVLAD and NetFV. These two encoding networks can perform a discriminative pooling network on the raw FC data to embed a large and varied number of cells as a fixed dimensional vector.

In some embodiments, the FC data matrix (for one sample, one tube, one chunk of a sample, or one chunk of a tube) can be encoded into the corresponding representation based on a FV-based encoding method, a VLAD-based encoding method, a NetFV encoding network, or a NetVLAD encoding network. The FV-based encoding method includes a FV encoding method which uses GMM based FV. The FV-based encoding method includes a FV-A encoding method which combines Fisher Vector and Autoencoder, and the Autoencoder was used in some AML MRD prediction studies. The VLAD-based encoding method includes a VLAD encoding method which uses the VLAD feature aggregation method. The NetFV encoding network is derived by applying the gradient updating function to the FV encoding method. The NetVLAD encoding network is derived by applying the gradient updating function to the VLAD encoding method.

### B.2.1. FV & NetFV Encoding

For FV, we compute the gradient vector of the FC sample X] (one tube of a patient) with respect to the parameter λ of the GMM density function p_{λ}(X). The gradient function is defined as:

$${∇}_{λ} log log p ( λ ) \qquad ( 1 )$$

The posterior probability of each chunk-level FC data x∈X for the k-th GMM component can be computed as:

$${γ}_{i} ( k ) = \frac{{π}_{k} {p}_{k} ( {x}_{i} )}{{\sum }_{{k}^{′} = 1}^{K} {π}_{k ′} {p}_{k ′} ( {x}_{i} )} \qquad ( 2 )$$

Then, we can derive the first-order and the second-order vectors by rewriting (1) as follows:

$${g}_{{μ}_{k}}^{X} = \frac{1}{T \sqrt{{π}_{k}}} {\sum }_{i = 1}^{T} {r}_{t} ( k ) ( \frac{{x}_{t} - {μ}_{k}}{{σ}_{k}} ) \qquad ( 3 )$$

$${g}_{{σ}_{k}}^{X} = \frac{1}{T \sqrt{2 {π}_{k}}} {\sum }_{i = 1}^{T} {r}_{t} ( k ) ( \frac{{( {x}_{t} - {μ}_{k} )}^{2}}{{σ}_{k}^{2}} - 1 ) \qquad ( 4 )$$

The first order and second order statistical estimation of gradients represents the direction for the learned distribution λ to better fit each sample X with the probability p_{λ}(X). g_{μ}_{ₖ}^{X} and g_{σ}_{ₖ}^{X}, are then concatenated as a vectorized FC representation.

Although FV can represent the chunk-level FC data as a vector using the pre-trained GMM parameters, the GMM training is independent of the classifier training. Here, we introduce NetFV to further improve the chunk-level representation in a supervised learning manner. NetFV estimates the final FV mathematical form using learnable parameters to embed the hematologic malignancy type information into the network. To reduce the network learning complexity, the GMM distribution is assumed to have equal weights and the p_{λ}(xᵢ) is written as

$${p}_{λ} ( {x}_{i} ) = \frac{1}{\sqrt{{( 2 π )}^{D}}} exp exp \{ - \frac{1}{2} {( {x}_{i} - {μ}_{k} )}^{T} {\sum }_{k}^{- 1} ( {x}_{i} - {μ}_{k} ) \} \qquad ( 5 )$$

Let

$${w}_{k} = \frac{1}{{σ}_{k}}$$

and bₖ=−μₖ and the cell posterior becomes:

$${γ}_{i} ( k ) = \frac{exp \{ - \frac{1}{2} {( {w}_{k} ⊙ ( {x}_{i} + {b}_{k} ) )}^{T} ( {w}_{k} ⊙ ( {x}_{i} + {b}_{k} ) ) \}}{{\sum }_{{k}^{′} = 1}^{K} exp \{ - \frac{1}{2} {( {w}_{k ′} ⊙ ( {x}_{i} + {b}_{k ′} ) )}^{T} ( {w}_{k ′} ⊙ ( {x}_{i} + {b}_{k ′} ) ) \}} \qquad ( 6 )$$

The ⊙ denotes the Hadamard product of matrices. Finally, the first-order and the second-order gradient vectors are also expressed by the differentiable parameters wₖ and bₖ.

$${R}_{{μ}_{k}} = {γ}_{i} ( k ) [ {w}_{k} ⊙ ( {x}_{i} + {b}_{k} ) ] \qquad ( 7 )$$

$${R}_{{σ}_{k}} = \frac{{γ}_{i} ( k )}{\sqrt{2}} [ {( {w}_{k} ⊙ ( {x}_{i} + {b}_{k} ) )}^{2} - 1 ] \qquad ( 8 )$$

Followed by the NetFV layer, we use the fully-connected network to predict the hematologic malignancy types for the supervised training. In the implementation shown in FIG. 2, the learnable terms in equations 7 and 8 are decoupled as weighting terms (a_{μ} and a_{σ}) and residual terms b_{μ}.

### B.2.2. FV & NetFV Encoding

Another distributional encoding approach, VLAD, also performs variable-length cell pooling based on the calculation of the sum of residuals for each GMM cell cluster. This method is an improvement of the traditional bag-of-words algorithm, and can better describe the relationship between the targeted cell and each learned cluster center. Given the same chunk-level FC data input X with varied cell numbers and the GMM cluster centers μₖ∈R^{K*D}, the weighted residual matrix V is computed as follows:

$$V ( k ) = {\sum }_{i = 1}^{N} {a}_{k i} * ( {x}_{i} - {μ}_{k} ) \qquad ( 9 )$$

where aₖᵢ is a binary indicator which denotes whether the k-th cluster is the closest cluster to the cell xᵢ. The summed residual vector describes the total distance between the cluster center and the whole cell data population assigned to it. The matrix V is normalized by column-wise L2-norm and an entire L2-norm after being flattened as the final fixed-length vector.

NetVLAD can encode the cells as a chunk-level representation by generalizing the approach as a trainable VLAD layer. To ensure that the parameters are differentiable, the cells are softly assigned to each cluster, using the normalized weight derived from the distance as aₖᵢ, instead of using a binary value (hard assignment).

$${a}_{k i} = \frac{exp ( - α {‖ {x}_{i} - {μ}_{k} ‖}^{2} )}{{\sum }_{k ′}^{K} exp ( - α {‖ {x}_{i} - μ {′}_{k} ‖}^{2} )} \qquad ( 10 )$$

where α is a positive constant that determines the decay rate of the whole exponential term. By replacing wₖ=2αμₖ and bₖ=−α∥μₖ∥², the soft assignment weight is derived as follows:

$${a}_{k i} = \frac{exp ( {w}_{k}^{T} {x}_{i} + {b}_{k} )}{{\sum }_{{k}^{′} = 1}^{K} exp ( {w}_{k}^{T} {x}_{i} + {b}_{k ′} )} \qquad ( 11 )$$

The NetVLAD layer is organized as the following form:

$$V ( k ) = {\sum }_{i = 1}^{N} \frac{exp ( {w}_{k}^{T} {x}_{i} + {b}_{k} )}{{\sum }_{{k}^{′} = 1}^{K} exp ( {w}_{k}^{T} {x}_{i} + {b}_{k ′} )} * ( {x}_{i} - {μ}_{k} ) \qquad ( 12 )$$

In (12) is normalized by L2-norm to generate a chunk-level FC representation. This end-to-end learning network is built with the NetVLAD layer followed by a deep feedforward network, to classify the hematologic malignancy categories. In this work, we experiment with the aforementioned SOTA distributional-based deep chunk-level embeddings (or chunk-level representations 252) for the task of sample-level FC data modeling.

### B.3. Chunk Ensemble Prediction

To aggregate the chunk-level representations, the present disclosure leverages a technique called the “implicit ensemble” approach. We extracted the chunk-level embeddings (e.g., the chunk representations 252) from the deep embedding network described in Section B.2. With an aggregation statistical function, such as the maximum, we can derive the aggregated representation (e.g., the representation 262 or the ensemble representation) from the chunk-level embeddings (e.g., the chunk representations 252) as input to the following dense layers (e.g., the dense layer shown in the ensemble prediction 260) for one sample (tube). Multiple aggregated representations for multiple samples (tubes) can concatenated for final classification or decision.

In the present disclosure, different kinds of aggregating functions 261 applied across chunks representations are used to keep the most salient value in each feature dimension across all chunks within a sample (e.g., a tube). For example, the aggregating functions 261 may include at least one of: a majority voting function, a maximum pooling function, a mean pooling function, a stochastic pooling function, or a median pooling function.

The chunk representations 252 for the same sample (e.g., tube) can be vector representations having the same number of feature dimensions. For one chunk representation 252, it has a first feature value for the first feature dimension, a second feature value for the second feature dimension, a third feature value for the third feature dimension, and etc. In the ensemble prediction 260, several chunk representations 252 are aggregated based on feature dimensions. For example, when the aggregating functions 261 is the majority voting function, the first value for the first feature dimension of the aggregated representation (e.g., the representation 262 or the ensemble representation) is determined by performing the majority voting function to the first values of the vector representations of the corresponding chunks (e.g., the chunk representations 252), the second value for the second feature dimension of the aggregated representation is determined by performing the majority voting function to the second values of the vector representations of the corresponding chunks, the third value for the third feature dimension of the aggregated representation is determined by performing the majority voting function to the third values of the vector representations of the corresponding chunks, and so on.

For example, when the aggregating functions 261 is the maximum pooling function, the first value for the first feature dimension of the aggregated representation (e.g., the representation 262 or the ensemble representation) is determined by performing the maximum pooling function to the first values of the vector representations of the corresponding chunks (e.g., the chunk representations 252), the second value for the second feature dimension of the aggregated representation is determined by performing the maximum pooling function to the second values of the vector representations of the corresponding chunks, the third value for the third feature dimension of the aggregated representation is determined by performing the maximum pooling function to the third values of the vector representations of the corresponding chunks, and so on. Therefore, the first value for the first feature dimension of the aggregated representation is the maximum value between the first values of the vector representations of the corresponding chunks, the second value for the second feature dimension of the aggregated representation is the maximum value between the second values of the vector representations of the corresponding chunks, the third value for the third feature dimension of the aggregated representation is the maximum value between the third values of the vector representations of the corresponding chunks, and so on.

The major advantage of the ensemble process (e.g., ensemble prediction 260) network proposed in the present disclosure is that the required memory consumption and computation power is limited, unlike explicit ensemble methods that duplicate multiple models for diverse outputs. With properly selected chunk sizes, each chunk should provide a sufficient discriminative capability using a small-sized samples of cells. The ensemble pooling process (e.g., ensemble prediction 260) is designed to keep the largest values in each feature dimension to maintain high discriminatory power and keep the feature dimension low.

### C. ARCHITECTURE DETAILS

The hyper-parameters are obtained via grid-search from a defined set. The deep embedding network described in Section B.2 is composed of either NetFV or Net VLAD modules with K clusters where K is selected from the values between 16 and 128 with a fixed step of 16. It has an output layer with [64, 128, 256, 1024] nodes. The following dense layer has [32, 64, 128, 256] nodes with ReLU or tanh activation functions. The dropout rate are searched in the range from 0 to 0.5, with a fixed step of 0.05. In the ensemble prediction network, a single softmax layer projects the aggregated representation to the prediction space which has the number of classes as the number of nodes. Using Adam optimizer, the network is optimized by choosing either cross-entropy or KL-divergence as the loss function, based on the best validation performance. The training process includes early stopping if the validation performance does not improve for continuous five iterations. The learning rate is adjusted from 0.001 to 0.01 using a log distribution sampler.

FIG. 3 is a flowchart of a method 300 according to some embodiments of the present disclosure. The method 300 may be a method of processing cytometric data. The method 300 includes operations 301, 303, 305, 307, 309, 311, 313, 315, 317, and 319.

In operation 301, one or more data matrices may be received. The one or more data matrices correspond to the cytometric data acquired from a flow cytometry. The number of the data matrices may correspond to the number of the samples (or the number of the specimens) inspected by a flow cytometry (e.g., the flow cytometry 220). In particular, one data matrix may be generated from a sample inspected by a flow cytometry. The number of the data matrices may correspond to the number of the tubes of a patient (or a case), in which antibody-fluorescence measurements may be performed to each of one or more tubes of a patient through a flow cytometry. In particular, one data matrix may be generated from a tube of a patient inspected by a flow cytometry. In some embodiments, one sample (or one specimen) corresponds to a tube of a patient.

In operation 303, one data matrix would be received. In some embodiments one data matrix would be selected from the one or more data matrices received in operation 301. One data matrix indicates a plurality of properties of a set of cells, and the set of cells correspond to the cells of a sample or a tube. In some embodiments, the plurality of properties of the set of cells correspond to the properties of the antibodies shown in one of the rows of Tube 1 through Tube 5 for the UPMC dataset. In some other embodiments, the plurality of properties of the set of cells correspond to the properties of the antibodies shown in one of the rows of Tube 1 through Tube 3 for the hema.to dataset. The data matrix in operation 303 may correspond to the data matrix 230 in FIG. 230. The data matrix in operation 303 may have D columns and T rows, in which D) is the number of properties inspected and Tis the number of cells inspected (or the number of cells in the set of cells).

In operation 305, the data matrix is divided or chunked into a plurality of submatrices. The submatrices may correspond to chunks 241 in FIG. 2. Operation 305 may correspond to the operation of chunking 240 in FIG. 2.

In operation 307, each of the plurality of submatrices is encoded into one corresponding vector representation. The plurality of submatrices are encoded into corresponding vector representations. The number of the plurality of submatrices may be associated with or identical to the number of the vector representations. In operation 307, the plurality of vector representations are acquired. The vector representation may correspond to the chunk representations 252 in FIG. 2 or the aforementioned chunk-level embeddings. Operation 307 may correspond to the chunk-level pooling network 250 in FIG. 2.

In operation 307, each of the submatrices may be transposed (corresponding to the operation of transposing 251 in FIG. 2). In some embodiments, since the submatrices are transposed, the number of rows of the submatrices may be correspond to the number of rows of the data matrix (i.e. the aforementioned variable)); after encoding, the number of feature dimensions of the vector representations may be correspond to or associated with the number of rows of the data matrix (i.e. the aforementioned variable D). The number of feature dimensions of each vector representation for the same data matrix (or the same sample or tube) may be identical. In some embodiments, one vector representation may be represented as a series of values, in which the number of values corresponds to the number of feature dimensions; the first value corresponds to the first feature dimension, the second value corresponds to the second feature dimension, and so on.

In operation 307, each of the submatrices may be encoded into one corresponding vector representation based on at least one of: a FV-based encoding method, a VLAD-based encoding method, a NetFV encoding network, or a NetVLAD encoding network. The FV-based encoding method includes a FV encoding method which uses GMM based FV. The FV-based encoding method includes a FV-A encoding method which combines Fisher Vector and Autoencoder, and the Autoencoder was used in some AML MRD prediction studies. The VLAD-based encoding method includes a VLAD encoding method which uses the VLAD feature aggregation method. The NetFV encoding network is derived by applying the gradient updating function to the FV encoding method. The NetVLAD encoding network is derived by applying the gradient updating function to the VLAD encoding method.

In operation 309, the plurality of vector representations are aggregated or ensembled into an ensemble representation. The ensemble representation may correspond to the representation 262 in FIG. 2.

In some embodiments, the ensemble representation may be aggregated or ensembled through aggregating the plurality of vector representations based on feature dimensions of the plurality of vector representations.

In some further embodiments, aggregating the plurality of vector representations based on the feature dimensions may be carried out through performing at least one of following functions to each feature dimension of the plurality of vector representations: a majority voting function, a maximum pooling function, a mean pooling function, a stochastic pooling function, or a median pooling function.

For example, when the aggregating functions 261 is the maximum pooling function, the first value for the first feature dimension of the ensemble representation is determined by performing the maximum pooling function to the first values of the first feature dimension of the vector representations, the second value for the second feature dimension of the ensemble representation is determined by performing the maximum pooling function to the second values of the second feature dimension of the vector representations, the third value for the third feature dimension of the ensemble representation is determined by performing the maximum pooling function to the third values of the third feature dimension of the vector representations, and so on. Therefore, the first value for the first feature dimension of the ensemble representation is the maximum value between the first values of the first dimension of the vector representations, the second value for the second feature dimension of the ensemble representation is the maximum value between the second values of the second feature dimension of the vector representations, the third value for the third feature dimension of the ensemble representation is the maximum value between the third values of the third feature dimension of the vector representations, and so on.

In operation 311, it is determined whether there is a next data matrix to be processed. For example, if only one data matrix is received in operation 301, it is determined that there is no next data matrix to be processed in operation 311. In some embodiments, if two data matrices are received in operation 301, it is determined that there is a next data matrix to be processed when the operation 311 is first executed, and operations 303, 305, 307, and 309 are performed again. Therefore, operations 303, 305, 307, 309, and 311 would be executed until all of the data matrices received in operation 301 are processed.

In operation 313, it is determined whether there are more than one ensemble representation are acquired in the previous operations. For example, if only one data matrix is received in operation 301, only one ensemble representation is acquired in the previous operations, and it is determined that there are not more than one ensemble representation in operation 313.

When it is determined that there are not more than one ensemble representations in operation 313, operation 315 is performed. In operation 315, the cytometric data would be classified based on the only one ensemble representation, wherein the only one ensemble representation is or generated based on the only one data matrix received in operation 301, and the only one data matrix corresponds to the cytometric data acquired from one sample or one tube through a flow cytometry.

In some embodiments, if more than one data matrices are received in operation 301, more than one ensemble representations are acquired in the previous operations, and it is determined that there are more than one ensemble representation in operation 313. For example, if five data matrices are received in operation 301, five corresponding ensemble representations are acquired in the previous operations.

When it is determined that there are more than one ensemble representations in operation 313, operation 315 is performed. In operation 317, the more than one ensemble representations are concatenated to acquire a concatenated representation. For example, if three ensemble representations are calculated or acquired from operations 301, 303, 305, 307, 309, and 311, the head (e.g., the first feature dimension) of the second ensemble representation is concatenated to the tail (e.g., the last feature dimension) of the first ensemble representation, the head (e.g., the first feature dimension) of the third ensemble representation is concatenated to the tail (e.g., the last feature dimension) of the second ensemble representation, and the concatenated representation is generated and acquired.

In operation 319, the cytometric data would be classified based on the concatenated representation, wherein more than one ensemble representations are calculated or generated based on the more than one data matrices received in operation 301, and the more than one data matrices correspond to the cytometric data acquired from more than one sample (or tube) through a flow cytometry. For example, if three ensemble representations are calculated or acquired from operations 301, 303, 305, 307, 309, and 311, the three ensemble representations are calculated or generated based on three data matrices received in operation 301, the three data matrices correspond to the cytometric data acquired from three samples or three tubes through a flow cytometry, and the cytometric data is classified based on the concatenated representation based on the three ensemble representations.

Upon classifying the cytometric data based on the corresponding concatenated representation (e.g., corresponding to operation 319) or ensemble representation (e.g., corresponding to operation 315), the type of hematologic malignancy can be classified, predicted, or detected with good accuracy.

### D. Experimental Setup

In the present disclosure, we carried out experiments on two datasets using five-fold patient-independent cross-validation: 80 percent was used for training and 20 percent for blind testing in each fold. We randomly selected 20 percent of the training data as a validation set for the tuning of hyper-parameters in each training fold. For all performance evaluation experiments, we calculated unweighted F1 score (UF1), weighted F1 score (F1), accuracy (ACC), area under ROC (receiver operating characteristic) curve (AUC), and unweighted average recall (UAR). The following sections include the classification results for discriminative capability and analyses for robustness and computational costs.

In Section E.1, we compare our results with those from the following list of methods. These algorithms are either from previous works or from the related methods.
Func: computing six statistical functional features;
SOM-CNN: using SOM to reduce FC data to two-dimensional inputs and CNN for classification (as used in M. Zhao et al., “Hematologist-level classification of mature B-cell neoplasm using deep learning on multi-parameter flow cytometry data,” Cytometry Part A, vol. 97, no. 10, pp. 1073-1080, 2020);
PCA-CNN: using PCA to reduce the data to two-dimensions for convolutional neural network;
GMM: using the averaged cell posterior vector of the specimen from the learned GMM (similar to the method proposed in B. Rajwa, P. K. Wallace, E. A. Griffiths, and M. Dundar, “Automated assessment of disease progression in acute myeloid leukemia by probabilistic analysis of flow cytometry data,” IEEE Trans. Biomed. Eng., vol. 64, no. 5, pp. 1089-1098 May 2017);
FV: using GMM based FV (as used in B.-S. Ko et al., “Clinically validated machine learning algorithm for detecting residual diseases with multicolor flow cytometry analysis in acute myeloid leukemia and myelodysplastic syndrome,” EBioMedicine, vol. 37, pp. 91-100, 2018, S. A. Monaghan et al., “A machine learning approach to the classification of acute leukemias and distinction from nonneoplastic cytopenias using flow cytometry data,” Amer. J. Clin. Pathol., vol. 157, no. 4, pp. 546-553, 2021, and J. Sánchez, F. Perronnin, T. Mensink, and J. Verbeek, “Image classification with the Fisher vector: Theory and practice,” Int. J. Comput. Vis., vol. 105, no. 3, pp. 222-245, 2013);
FV-A: combining Fisher Vector and Autoencoder (the Autoencoder was used in the AML MRD prediction study, J. Li, Y. Wang, B. Ko, C. Li, J. Tang, and C. Lee, “Learning a cytometric deep phenotype embedding for automatic hematological malignancies classification,” in Proc. 41st Annu. Int. Conf. IEEE Eng. Med. Biol. Soc., 2019, pp. 1733-1736);
VLAD: using the VLAD feature aggregation method (as used in H. Jégou, M. Douze, C. Schmid, and P. Pérez, “Aggregating local descriptors into a compact image representation,” in Proc. IEEE Comput. Soc. Conf. Comput. Vis. Pattern Recognit., 2010, pp. 3304-3311);
N-VLAD: using NetVLAD as described in Section B.2 of the present disclosure;
N-FV: using NetFV as described in Section B.2 of the present disclosure;
Ev-X: using our proposed method (e.g., the framework 200 or the method 300), in which the majority voting function is used in the ensemble prediction 260 (e.g., operation 309);
Ef-X: using our proposed method (e.g., the framework 200 or the method 300), in which the maximum pooling function is used in the ensemble prediction 260 (e.g., operation 309);

These comparison approaches can be generally separated into non-GMM based algorithms, GMM-based algorithms and ensemble (chunking-for-pooling) methods. Func, SOM-CNN (Self-Organizing Map-Convolutional Neural Networks), and PCA-CNN (Principal Component Analysis-Convolutional Neural Networks) are non-GMM based algorithms. They use simple statistical functions (Func) to condense the large cell vectors or reduce the dimensions with algorithms such as SOM and PCA. SOM-CNN is a recently proposed deep framework that takes advantage of the conventional SOM algorithm in the automatic FC modeling domain and introduces CNN to process image-like downsampled data. PCA-SOM is another baseline included to compare the different dimensional reduction approaches due to the widely used PCA approach for FC visualization.

There is also a series of distributional learning algorithms, including GMM, FV, FV-A, VLAD, N-VLAD, and N-FV. Traditional approaches such as VLAD and FV explicitly calculate the residual and gradient by means of learned GMM parameters. FV-A adds an additional autoencoder to transform the feature space. The two end-to-end networks, NetVLAD and NetFV directly use supervised network learning for a discriminative GMM-based representation.

In our chunking-for-pooling framework, we use chunks as the network input and further perform chunk aggregation. Therefore, we compare two kinds of aggregation approaches, Ev-X and Ef-X, to implement chunk aggregation for the sample-level recognition. The voting strategy of Ev is the majority voting strategy while Ef builds a fully connected network to classify the chunk representation aggregated by the maximum pooling function. The “X” in Ev-X and Ef-X can be either N-VLAD or N-FV, which represents the choice of deep embedding network architecture. In Table IV (dividing into Table IV (a) and Table IV (b)), we choose the best-performing algorithm on each dataset for the ensemble (chunking-for-pooling) framework (e.g., corresponding to the framework 200 or the method 300) comparison.

Table IV (dividing into Table IV (a) and Table IV (b)) shows the hematologic malignancy classification results on the UPMC dataset and the hema.to dataset.

TABLE IV (a)

| | | SOM- | PCA- | | | |
|---|---|---|---|---|---|---|
| | Func | CNN | CNN | GMM | VLAD | FV |
| UPMC | | | | | | |
| UF1 | 0.836 | 0.529 | 0.609 | 0.742 | 0.626 | 0.885 |
| WF1 | 0.864 | 0.653 | 0.745 | 0.823 | 0.716 | 0.894 |
| ACC | 0.864 | 0.674 | 0.766 | 0.832 | 0.725 | 0.895 |
| AUC | 0.960 | 0.801 | 0.897 | 0.905 | 0.831 | 0.975 |
| UAR | 0.832 | 0.547 | 0.614 | 0.728 | 0.610 | 0.865 |
| hema.to | | | | | | |
| UF1 | 0.783 | 0.569 | 0.670 | 0.728 | 0.419 | 0.786 |
| WF1 | 0.847 | 0.741 | 0.808 | 0.825 | 0.652 | 0.849 |
| ACC | 0.859 | 0.756 | 0.815 | 0.837 | 0.704 | 0.856 |
| AUC | 0.941 | 0.805 | 0.847 | 0.869 | 0.747 | 0.950 |
| UAR | 0.751 | 0.550 | 0.650 | 0.713 | 0.403 | 0.770 |

TABLE IV (b)

| UPMC | FV-A | N-VLAD | N-FV | Ev-N-FV | Ef-N-FV |
|---|---|---|---|---|---|
| UF1 | 0.852 | 0.877 | 0.899 | 0.910 | 0.923 |
| WF1 | 0.885 | 0.897 | 0.922 | 0.927 | 0.934 |
| ACC | 0.887 | 0.898 | 0.923 | 0.927 | 0.934 |
| AUC | 0.953 | 0.976 | 0.982 | 0.986 | 0.987 |
| UAR | 0.829 | 0.857 | 0.880 | 0.924 | 0.923 |

| hema.to | FV-A | N-VLAD | N-FV | Ev-N-VLAD | Ef-N-VLAD |
|---|---|---|---|---|---|
| UF1 | 0.734 | 0.810 | 0.806 | 0.817 | 0.851 |
| WF1 | 0.835 | 0.861 | 0.854 | 0.865 | 0.878 |
| ACC | 0.841 | 0.870 | 0.856 | 0.870 | 0.877 |
| AUC | 0.922 | 0.959 | 0.943 | 0.949 | 0.962 |
| UAR | 0.728 | 0.795 | 0.784 | 0.783 | 0.850 |

### E. Classification Results

In this Section, we report the results of classification experiments including comparisons of the representation learning algorithms, the ensemble (chunking-for-pooling) approach comparison, and error analysis. We include state-of-the-art algorithms and ensemble (chunking-for-pooling) approaches for FC data modeling in Section III-B1, and analyze the effect of different parameter choice such as different chunk quantities, blast percentage, and hematologic malignancy categories on the classification outcomes.

### E.1. Comparison of Representation Learning Algorithms

The present disclosure compares the different algorithms using FC data and shows results in Table IV. We first compare the network architectures and the encoding approaches excluding the ensemble (chunking-for-pooling) results (Ev-X and Ef-X) in Table IV. It is observed that N-FV achieved the highest four-class hematologic malignancy type classification performance on the UPMC dataset with 89.9% UF1, 92.3% ACC and 88.0% UAR. N-VLAD outperformed the other algorithms in the five-class hematologic type classification on the hema.to dataset. Generally, excluding the ensemble (chunking-for-pooling) results (Ev-X and Ef-X), N-FV and N-VLAD have better performance than others.

We included widely-used encoding algorithms as baselines. For example, Func achieved 83.2% UAR and 75.1% on UPMC and hema.to datasets, respectively. The recently proposed SOM-CNN achieved 52.9% UF1, 67.4% ACC, and 54.7% UAR on the UPMC dataset and 56.9% UF1, 75.6% ACC, and 55.0% UAR on the hema.to dataset. Although the results of the hema.to dataset were evaluated based on their partially released data, our comparison on two clinically collected datasets for complete validation overcame the problems of the past studies, which usually lacked comprehensive comparisons of algorithm across different data cohorts. When we compared SOM to PCA, the same CNN architecture was used, and we obtained better results for PCA-CNN. The performance of PCA-CNN was inferior to that of Func with −22.7% UF1, −9.8% ACC, and −21.8% UAR on the UPMC dataset and −11.3% UF1, −4.4% ACC, and −10.1% UAR on the hema.to dataset. Compared to dimensional reduction approaches (often reduced to 2-D plots), using statistical functions (Func) is more effective in summarizing statistical properties in the FC data.

The GMM-based algorithm is a major distributional branch in modeling FC data, and its corresponding encoding approaches have been shown to be the SOTA in the AML and ALL MRD detection tasks. We compared these approaches with the aforementioned baselines to provide a comprehensive computational framework validation (Table IV). GMM obtains a low accuracy, in that the oversimplified averaged posterior vectors only include a crude probabilistic summarization of the sample distribution. In contrast, VLAD, FV, N-VLAD and N-FV are algorithms designed upon GMM which compute complex functions to describe the relationship between the cluster and each individual data sample as high-dimensional vectors. VLAD and FV are both approaches without the use of deep network learning. We observe that FV outperformed VLAD with its 88.5% UF1, 89.5% ACC, and 86.5% UAR on the UPMC dataset and 78.6% UF1, 85.6% ACC, and 77.0% UAR on the hema.to dataset. The main reason is that VLAD only represents the sample distance to the cluster center, with a hard assignment to a specific cluster. The unassigned clusters have no contribution to the representation and the residual calculation only considers the cluster centers, without the covariance of the GMM distribution. In contrast, FV estimates the gradients in terms of the mean and covariance parameters of the GMM, and results in higher dimensional representations. Therefore, the FV representations include a more complete description of the data by simultaneously taking into account the cluster center and covariance in terms of each sample point. FV-A uses an autoencoder to transform feature space for FV which has been adopted for the MRD detection task. However, the low accuracy imply that the transformation would only be suitable in the AML MRD recognition task rather than in the multiclass recognition tasks in this work.

These compared representations were derived in an unsupervised manner, independent of the classifier. By leveraging end-to-end networks, N-VLAD and N-FV improve the performance of VLAD and FV by using supervised representation learning. On the UPMC dataset, N-FV achieved higher performances than FV by 1.58% for relative UF1, 3.13% for relative ACC, and 1.73% for relative UAR. Similarly, the performance of N-VLAD was better than that of VLAD by 25.1% UF1, 17.3% ACC, and 24.7% UAR. The improvements were also shown on the hema.to dataset. UF1 and UAR were improved in terms of N-FV versus FV and improvements of 39.1% UF1, 16.6% ACC, and 39.2% UAR were obtained for N-VLAD over VLAD. The significant difference between N-VLAD and VLAD stems from the hard assignment design in VLAD. When N-VLAD adopts soft-assignment and derives the parameters through learning, the performance can be comparable to that of N-FV. The deep network produced significant gains for both N-FV and N-VLAD, and achieved the best performance across the five different metrics in Table IV

Overall, the advantages of N-VLAD and N-FV are their powerful discriminatory capability with an end-to-end optimization strategy, while other distributional learning approaches, such as VLAD and FV, are not discriminatively learned in an end-to-end manner. However, these deep learning-based approaches need GPU for training which can be a disadvantage when the computational resources are limited. The distributional approaches have advantages over the traditional statistical approaches and dimension reduction approaches in terms of model accuracy. Several previous studies have used GMM to visualize the pattern of FC data and thus another advantage of the distributional learning approach is its ability to provide intuitive visualization for clinicians or inspectors. The disadvantage of N-VLAD and N-FV is that it requires more complicated processes to provide clinicians or inspectors with an intuitive interpretation. Therefore, we performed a series of analyses to elaborate the model behavior.

### E.2. Comparison of Ensemble Methods

In the present disclosure, the ensemble methods may be corresponding to the chunking-for-pooling framework (e.g., the framework 200) or the method 300.

The best-performing models on the UPMC dataset and the hema.to dataset are N-FV and N-VLAD, respectively. Therefore, we applied the chunking-for-pooling framework using the best-performing networks. Ef-N-FV and Ef-N-VLAD consistently produced superior performance of the Ef ensemble strategy over the other algorithms on the UPMC dataset and hema.to dataset. Ef-N-FV achieved the highest four-class performance with 92.3% UF1, 93.4% ACC, and 92.3% UAR, which were 2.4% UF1, 1.1% ACC, 4.3% UAR improvements over N-FV on the UPMC dataset. Similarly, Ef-N-VLAD achieved the best five-class performance with 85.1% UF1, 87.7% ACC, and 85.0% UAR on the hema.to dataset. Ev-N-FV and Ef-N-VLAD with the chunking and pooling process obtained performance gains from N-FV and N-VLAD. The improvements using the ensemble approaches show the advantage on prediction capability and the resulting parameters such as chunk size are discussed in Section F.2.

Comparing the two ensemble strategies, Ev and Ef, the advantage of Ef was relatively minor on the UPMC dataset, but more apparent on the hema.to dataset. In the different evaluation metrics, the major improvements using Ef were on UF1 and UAR. Ef-N-FV had 2.67% and 4.89% relatively higher UF1 and UAR than N-FV on the UPMC dataset and Ef-N-VLAD had 5.06% and 6.92% relatively higher UF1 and UAR than N-VLAD on the hema.to dataset. These results show that Ef can better handle the imbalanced class issue. When the model is less discriminative, the model tends to predict the class with more samples which leads to lower UF1 and UAR. Ef generally outperforms Ev in that the newly learned layer can adapt the optimized weights for the maximally aggregated samples where the voting approach used in Ev depends primarily on the individual chunk predicting results leading to a sub-optimal classification performance.

### E.3. Comparison of Network Parameters

The number of clusters for N-VLAD and N-FV is selected by grid-search during training with the validation data set. Hence, we investigated the selected number of clusters in each fold to gain insights into the cell diversity. Both N-FV and N-VLAD achieved the best performance by specifying 48 clusters in the UPMC dataset, while they produced slightly different results on the hema.to dataset. The most suitable cluster number varied across folds on the hema.to dataset and N-FV tended to perform well with larger numbers of clusters (112). Although N-VLAD outperformed N-FV on the hema.to dataset, most of the folds use 16 clusters on the best validated model. Therefore, the results suggested that higher cluster size did not necessarily give rise to better performance.

According to the previous studies, blood cells can be divided into several lineages, such as red blood cells, lymphocytes, and myeloid cells [40]. The cells also include various different maturation stages of hematopoiesis. The types of hematologic malignancies are classified in large part based on the altered cell types and distributions that can be recognized by the FC pattern of antigen expression. Rough cell classification based on a tree-like hierarchy identifies dozens of subtypes across lineages. Intuitively, this number of cell type categories is the latent factor affecting the cluster size. The variation in cluster size can depend on the data distribution in each fold. If the population of a specific cell type is not distinguishable among different hematologic malignancies, the cluster would be merged with other similar cells from the same lineage during optimization of supervised learning. N-FV tended to obtain larger clusters because it calculates both the first and second order statistics and observes more cell level details. Overall, the largest selected number of cluster size was 112, although we searched up to 128. This cluster size range would be sufficient to represent the cell patterns in the hematologic type classification. The lineage commitment of some hematopoietic stem cells cannot be easily identified which would influence the automatically learned cluster size.

### E.4. Error Analysis

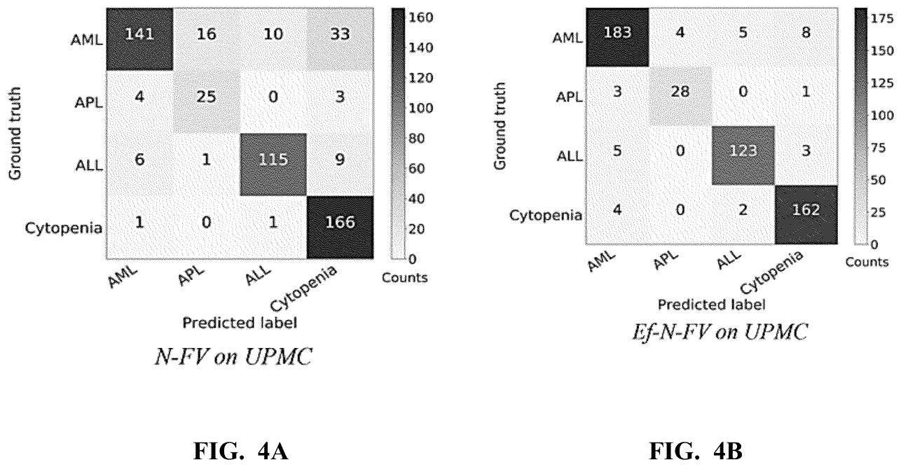

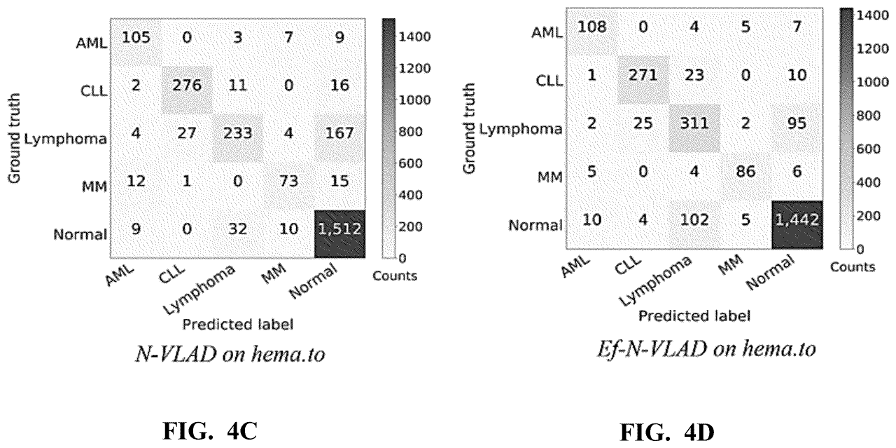

In this Section, we analyze the results of the best performing models found in the previous Section. First, we examined the confusion matrix in each of FIGS. 4A to 4D using the model with and without applying the chunking-for-pooling ensemble framework. FIGS. 4A to 4D are the confusion matrices using the best performed network architecture and the Ef approach on the UPMC and hema.to datasets.

In the first analysis, the major misclassification samples of N-FV on the UPMC dataset were from the AML class and our proposed Ef-N-FV could reduce the misclassification of AML. Specifically, N-FV wrongly predicted 59 AML samples as other classes, while Ef-N-FV only misclassified 17 AML samples. We observed that the multiclass hematologic malignancy classification often suffered more from the class with large number of samples compared to others, due to the diversity of the large size of samples. With our proposed framework, all of the cell diversity is included into chunks and aggregated in the final model. The ability to capture the large sample variability can better represent the class.

On the hema.to dataset, the prediction on AML was also improved by our proposed Ef approach. The model tended to predict the lymphoma class correctly, especially those samples misclassified as the normal classes. The 95 lymphoma samples misclassified as normal class contained mostly from MBL and LPL which are shown in Table V. MBL, a non-cancer precursor of CLL, sometimes demonstrate normal B-lymphocyte counts or mild abnormality introducing uncertainty to the diagnosis. The diagnosis of LPL based on morphology and immunophenotype is complex because it includes a large variety of admixed cell types (e.g. plasma cells, plasmacytoid cells, lymphocytes and mast cell), and LPL has been usually described as a diagnosis based on exclusion of other small B cell lymphomas. Thus, MBL and LPL are inherently more difficult to distinguish from other subtypes. Increasing the number of samples within these categories may improve the classification of these subtypes. The prediction of MM was also improved from 73 correctly classified samples to 86 correctly classified samples. However, the misclassification between the lymphoma class and the normal class still occurred and Ef-N-VLAD was prone to predict normal samples as lymphoma. When the chunks included more information to describe the diverse lymphoma subtypes, the model was more likely to correctly predict the lymphoma class. This publicly available hema.to dataset only includes a small portion of the whole data cohort. Therefore, the misclassification between the lymphoma and the normal class would be mitigated when learning with a larger dataset.

Table V shows the misclassified sample distribution of lymphoma subtype.

| Subtypes | HCL | FL | MBL | MCL | MZL | PL | LPL |
|---|---|---|---|---|---|---|---|
| Total | 77 | 24 | 60 | 38 | 116 | 36 | 84 |
| Ef-N-VLAD | 11 | 9 | 39 | 8 | 22 | 9 | 30 |
| N-VLAD | 29 | 18 | 49 | 10 | 36 | 10 | 50 |

### F. FRAMEWORK ANALYSIS

In this Section, we analyze the effects of applying our proposed framework for FC data modeling to several different aspects other than the classification performance. We illustrate the variability of model prediction results and the advantage of including all the cells without downsampling in Section F.1. Then, we analyze the effect on the chunk size and the total number of chunks, to further demonstrate the robustness of our proposed framework in Section F.2. Finally, we report the computational resources consumed by the model deployment in Section F.3.

### F.1. Investigation of Model Prediction Variability

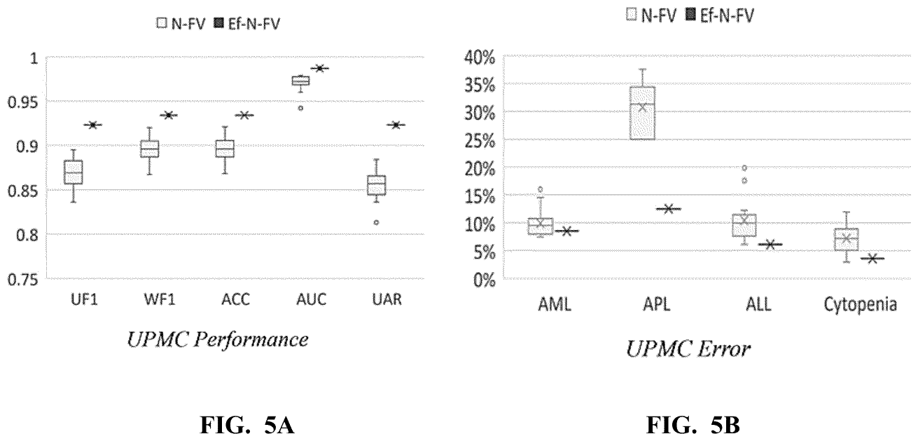

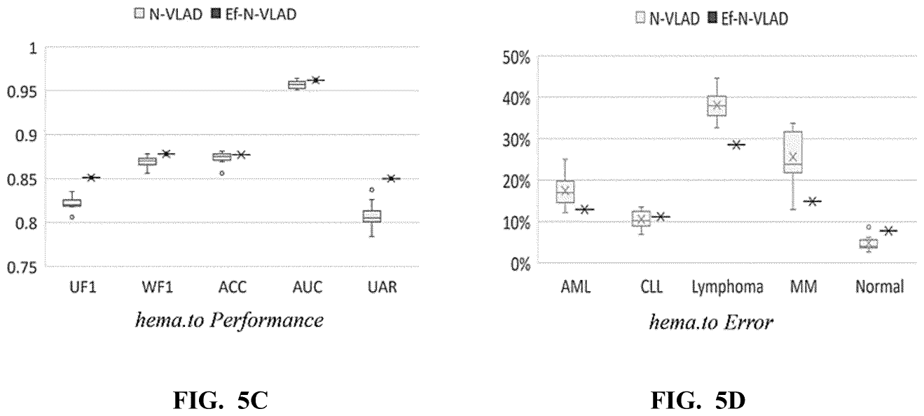

In these experiments, we examined the variability of the model prediction results using a downsampling strategy in comparison to our proposed “chunking-for-pooling” ensemble framework. Our proposed approach has the advantage of enabling model classification without dropping any cell. Therefore, this experiment can help show the extent to which robustness improvements can be obtained by our proposed method. The downsampled approach using N-FV or N-VLAD was implemented for 17 times to derive 17 independent results. We produced the box plots of the accuracy and the prediction outcome distributions across these randomly replicated experiments, as shown in FIGS. 5A to 5D. For Ef-N-FV and Ef-N-VLAD on the UPMC and hema.to datasets, there was only an exact value in each bar because the ensemble approach use all available cells without downsampling.

FIGS. 5A to 5D show the performance and error distributions in the downsampling (e.g., the legends of N-FV and N-VLAD) and chunking-for-pooling (e.g., the legends of Ef-N-FV and Ef-N-VLAD) experiments on the UPMC and hema.to datasets.

In FIGS. 5A to 5D, the mean values of UF1, WF1, ACC, AUC, and UAR using N-FV and N-VLAD all deviated from the results in Table IV. The two highest standard deviations, of UF1 and UAR are 0.0163 and 0.0165 on the UPMC dataset. The two highest standard deviations were 0.0070 and 0.0123 for UF1 and UAR on the hema.to dataset. If we approximate the confidence interval to cover 95% of the resampling outcomes, the model will lead to an interval with four times the standard deviation on the UPMC dataset: 6.52% for UF1 and 6.60% for UAR. The variability was smaller but still worth noting in the hema.to dataset. Its 95% confidence interval was 4.92% which not only influences the algorithm comparison, but more importantly introduces a serious concern about the validity of the clinical model. We performed Student's t-tests (FIGS. 5A to 5D) and found that all results of Ef-N-FV and Ef-N-VLAD on the different metrics were significantly higher than those of N-FV and N-VLAD (p-value<10⁻³) except for ACC in the hema.to dataset (p-value=0.055).

With respect to the samples in each hematologic malignancy class, we also report the misclassified sample ratio using the same box plot figures in FIGS. 5A to 5D which indicate the risk of the robustness issue. In the UPMC dataset, observing the mean percentage of the classification error of each class, the deviation from Ef-N-FV is significant. The standard deviation values of the misclassified numbers were 2.31%, 4.32%, 3.47% and 2.53% and the exact sample numbers could be obtained by multiplying the percentage by the total sample number of each class in Table II. The disadvantage of model robustness using naïve downsampling was also obvious when examining each class separately. As N-FV achieved excellent performances (0.923 ACC and 0.880 UAR), the prediction error and the explainable model predicting behavior becomes the key for clinical applications. For example, the tendency of N-FV to misclassify AML samples as cytopenia or APL in FIGS. 4A to 4D can lead to different clinical effects. The diminished prediction variability can establish a basis for further interpretation of model behavior. In the hema.to dataset, we observed that the distribution of AML, Lymphoma, and MM using N-VLAD were scattered in a wide range, and introduced significantly more error than Ef-N-VLAD. While the error of CLL and the normal classes had fewer errors, the variability of N-VLAD was still observable.

The robustness on the classification error on lymphoma in comparison with the Ef-N-VLAD and N-VLAD echoes the significant improvement on lymphoma mentioned in Section E.4. In fact, lymphoma contains different subtypes with few samples described in Section A. The limited amount of lymphoma subtype data makes the learned latent distribution less representative, and therefore is vulnerable to any slight change in the training samples. We infer that those hematologic malignancy types would suffer more variability in the downsampling process if the natural diversity was larger. This experiment illustrates that downsampling randomizes the model behavior and thus researchers cannot tell which samples can be correctly predicted. The deviation of the model performance in different downsampling trials also leads to unacceptable uncertainty in real world usage. In contrast, our proposed framework (e.g., the chunking-for-pooling framework, the framework 200, or the method 300) accounts for all the cells for a deterministic predicting result without the variability in every resampling experiment. We believe that this advantage is a crucial breakthrough in promoting the application of automatic classification systems to achieve clinical grade.

### F.2. Effects of Chunk Size and Chunk Quantity

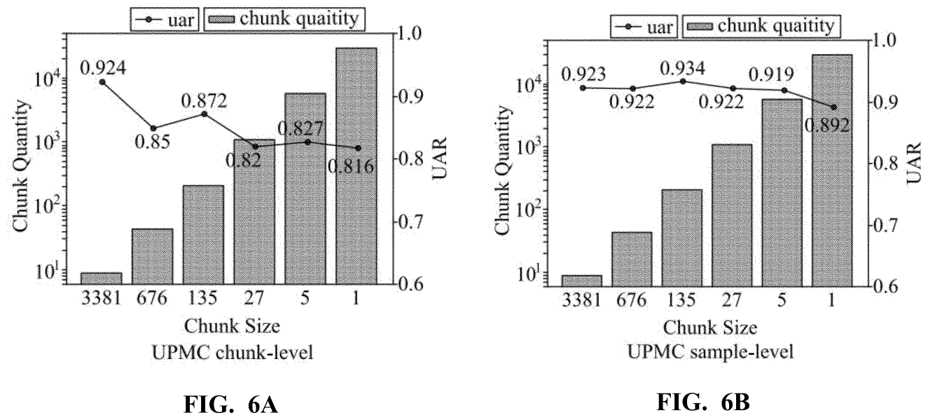

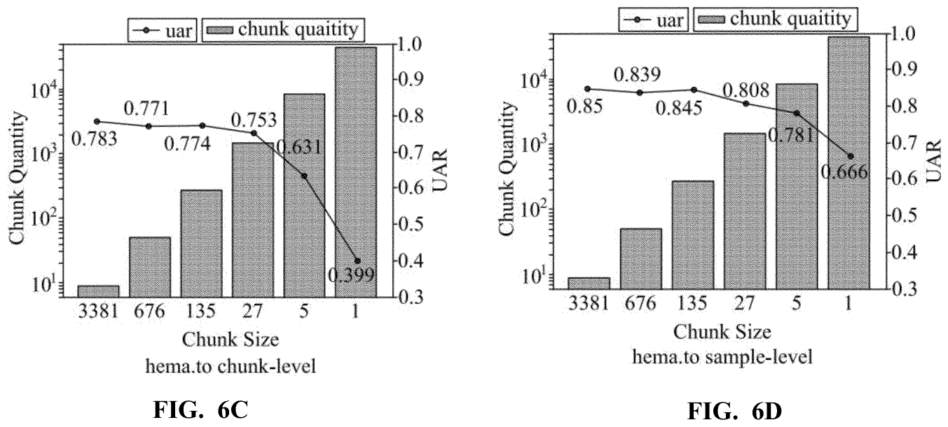

In this Section, we further analyze the effect of chunk size in our proposed framework (e.g., the chunking-for-pooling framework, the framework 200, or the method 300). The chunking process (e.g., the operation of chunking 240 or operation 305) acts as the key step to enable efficient GPU memory usage and the improvement of accuracy. The chunk size can be simply determined by the available GPU memory size, and we explored the performance change with different chunk sizes. The FC data from the UPMC and hema.to datasets mostly include a standard number of collected cells. Therefore, the change in chunk size will change the chunk quantity. That is, a small chunk size causes only a few cells to be used as a training sample for the deep network, while the total number of training samples become larger. In FIGS. 6A to 6D, we examine the outputs of the chunk-level pooling network 250 and the ensemble prediction 260 in our framework 200 to report both the chunk-level and the sample-level performances. The evaluation of chunk-level prediction was achieved by duplicating the sample-level label for the chunks and the sample-level performance was evaluated after the chunk-level outputs were aggregated by Ef. We selected UAR as the metric to avoid bias from imbalanced classes.

FIGS. 6A to 6D shows the results of our proposed framework (e.g., the chunking-for-pooling framework, the framework 200, or the method 300) with different chunk quantity and chunk size on the UPMC and hema.to datasets. Chunk-level results denote the results before ensemble aggregation (e.g., the ensemble prediction 260 or operation 309) and the sample-level results are after ensemble aggregation.

For the four-class hematologic malignancy classification on the UPMC dataset, the chunk-level prediction using 3381 cells in a chunk achieves 92.4% UAR, as shown in FIG. 6A and 92.3% UAR, as shown in FIG. 6B. Although the chunk-level performance declines when using smaller chunk size (e.g., 676, 135, and 27), the sample-level performance remained at a similar level. Because not every cell from a sample is an abnormal cell, some chunks would provide less information about the abnormal pattern of the hematologic malignancy. The chunk error can be eliminated by Ef, which optimizes the aggregated maximum values of the representation. When using a chunk size of 3381, there are eight chunks on average to perform the final pooling stage. By adjusting the chunk size, we found that the highest sample-level UAR was 93.4%, using 135 cells in a chunk. After this point, decreasing chunk size from 27 to only 1 cell per chunk gradually led to performance degradation. The performance drop from chunk size 5 to 1 is relatively obvious at the sample-level. The reason why single cells can produce the discriminative power can be attributed to the high ratio of abnormal leukemia cells or lymphocytes with different expression from the normal cells. Compared to the deterioration of chunk-level classification results, the sample-level results using Ef-N-FV had a relatively minor decrease in UAR. Using a single cell in a chunk still achieved 89.2% UAR which was 3.1% lower than the UAR using 3381 cells in a chunk. A sufficient pooling capacity for a supervised learning deep network was achieved when the chunk quantity increased to 29339. We concluded that the representations extracted using the Ef still preserved the cell characteristics, even though the chunk-level prediction was inaccurate.

For the five-class recognition task on the hema.to dataset, a decrease in chunk size did not help performance at the chunk level. The UAR dropped from 78.3% to 39.9% as the chunk size from 3381 to 1. Although sample-level results using 3381, 676 and 135 chunk size did not differ from each other significantly, the declining trend was still obvious using the chunk size from 27 to 1. The single cell chunk size achieved 66.6% sample-level UAR still shows the effectiveness of Ef to aggregate chunks with only 39.9% chunk-level UAR. Comparing the four-class and five-class tasks on the two datasets, the performance degradation is more significant in the hema.to dataset to predict five different hematologic malignancy types. The FC data in the hema.to dataset include more cells than the UPMC dataset and thus we can infer that the high performance on differentiating the five-class hematologic malignancy types would rely on particular cells. If the cells are rare but important, a small chunk size would give rise to a large number of incompletely observed chunks and dilute the information in the final pooling step. Once the task contains classes belonging to the same lineage or similar cell distributions, more cells will be needed in a single chunk to preserve the discriminative ability.

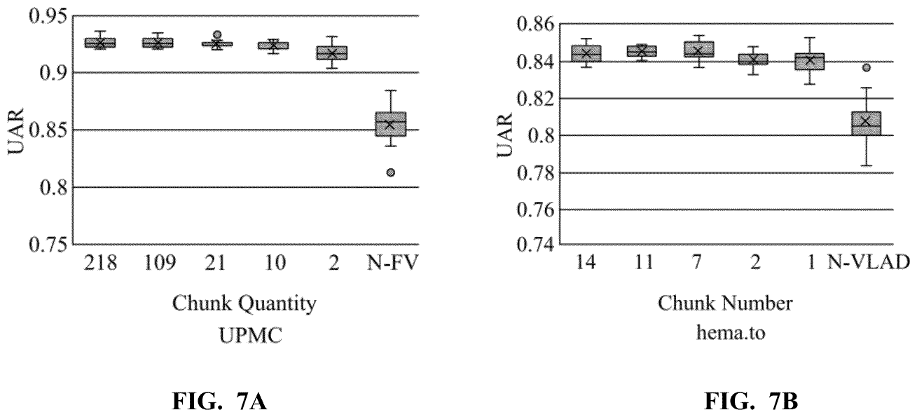

To further explore the robustness of the chunking process, we investigated the effect of reducing the chunk quantity in the best-performed chunk size condition shown in FIGS. 6A to 6D. The implementation was similar to that of the robustness experiment in Section F.1 and used UAR as the metric. We replicated the training and testing several times and obtained a distribution for each chunk quantity condition. Based on the data shown in FIGS. 6A to 6D, we used 135 and 3381 as the chunk sizes for the UPMC and hema.to datasets and the results of decreased chunk quantity are shown in FIGS. 7A and 7B. We observed that the model still achieved high performance as the chunk quantity reduced while the standard deviation was relatively large when including only a very few chunks. The situation reflects the robustness of Ef, which can eliminate the error through ensemble chunk aggregation. Compared to N-FV and N-VLAD which downsamples the FC data without the chunking and pooling process, Ef-N-FV and Ef-N-VLAD using any chunk quantity can achieve significantly better UAR (p-value<10⁻³). These results indicate that our proposed framework (e.g., the chunking-for-pooling framework, the framework 200, or the method 300) is robust even though the data have been downsampled.

FIGS. 7A and 7B shows the resulting distributions with the reduction of chunk quantity using our proposed framework (e.g., the chunking-for-pooling framework, the framework 200, or the method 300) are reported. The chunk size is fixed to be the best performed size from FIGS. 6A to 6D.

### F.3. Computational Resource Analysis

To evaluate the usability of the framework disclosed in the present disclosure, we focused on the computational consumption. Using the chunking process, the model input size can be reduced by a factor of the total number of chunks. Consequently, the memory used was much smaller than that used by the model stacked with the full-size FC data input. For example, the inputs are divided into around nine chunks in the UPMC datasets. The model size was reduced by 4.4 times compared with N-FV and Ef-N-FV. Similarly, the model size was reduced by 1.53 times in the hema.to dataset, as the FC data were divided into an average of 14 chunks. The model size using our proposed framework (e.g., Ef) can reduce the memory requirement. We use a Geforce RTX 2080Ti with 11 GB GPU memory to implement the framework. That is, the maximally allowed cell number in a chunk of the raw FC data with 37 antibody-fluorescent was 70,000 in an RTX 2080Ti for N-FV. This allowed cell number is usually insufficient in several clinical FC measurement scenarios. Another factor in the usability is the computational time. In comparisons between the model using the downsampling strategy and the model using our proposed chunking-for-pooling framework, the training computational time per epoch was 0.242 seconds and 0.109 seconds for N-FV and Ef-N-FV, respectively, on the UPMC dataset when using a batch size of 64 and a chunk size of 3381. Similarly, the training time per epoch was 1.143 seconds and 1 seconds for N-VLAD and Ef-N-VLAD on the hema.to dataset. Using the same setting in the testing phase, N-FV and Ef-N-FV took 0.202 seconds and 0.1 seconds for each batch with 16 samples on the UPMC dataset. N-VLAD and Ef-N-VLAD took 1.095 seconds and 1.007 seconds for each batch on the hema.to dataset. The “chunking-for-pooling” framework disclosed in the present disclosure (e.g., the framework 200 or method 200) further provided an acceleration of the training and testing process.

### G. CONCLUSION

The present disclosure proposes a chunking-for-pooling framework (e.g., the framework 200 or method 300) to improve robustness and prediction performance for automatic hematologic malignancy classification using FC data. The framework of the present disclosure addresses the issues of suboptimal unsupervised representation and incomplete data usage in prior research. Specifically, the framework of the present disclosure enables the use of very large FC cell-level data (complete cell numbers) by segmenting (e.g., chunking or dividing) the FC data matrix into chunks and further aggregating (or ensembling) representations of the chunks for sample-level prediction. This approach not only outperformed the other algorithms, but also mitigated the issue of robustness during the traditional downsampling step. Our further analyses investigated different factors, such as total chunk quantity, blast percentage, and disease types that can lead to misclassification. The quantitative statistics of downsampling results and the experiment of reducing the chunk size show the robustness of our model. Sample-level performance of the framework of present disclosure remains high even if the chunk-level prediction is not accurate. The reduced computational consumption and faster training speed are highly desirable for real clinical usability.

The scope of the present disclosure is not intended to be limited to the particular embodiments of the process, machine, manufacture, and composition of matter, means, methods, steps, and operations described in the specification. As those skilled in the art will readily appreciate from the disclosure of the present disclosure, processes, machines, manufacture, composition of matter, means, methods, steps, or operations presently existing or later to be developed that perform substantially the same function or achieve substantially the same result as the corresponding embodiments described herein may be utilized according to the present disclosure. Accordingly, the appended claims are intended to include within their scope processes, machines, manufacture, and compositions of matter, means, methods, steps, or operations. In addition, each claim constitutes a separate embodiment, and the combination of various claims and embodiments are within the scope of the disclosure.

The methods, processes, or operations according to embodiments of the present disclosure can also be implemented on a programmed processor. However, the controllers, flowcharts, and modules may also be implemented on a general purpose or special purpose computer, a programmed microprocessor or microcontroller and peripheral integrated circuit elements, an integrated circuit, a hardware electronic or logic circuit such as a discrete element circuit, a programmable logic device, or the like. In general, any device on which resides a finite state machine capable of implementing the flowcharts shown in the figures may be used to implement the processor functions of the present disclosure.

An alternative embodiment preferably implements the methods, processes, or operations according to embodiments of the present disclosure on a non-transitory, computer-readable storage medium storing computer programmable instructions. The instructions are preferably executed by computer-executable components preferably integrated with a network security system. The non-transitory, computer-readable storage medium may be stored on any suitable computer readable media such as RAMs, ROMs, flash memory, EEPROMs, optical storage devices (CD or DVD), hard drives, floppy drives, or any suitable device. The computer-executable component is preferably a processor, but the instructions may alternatively or additionally be executed by any suitable dedicated hardware device. For example, an embodiment of the present disclosure provides a non-transitory, computer-readable storage medium having computer programmable instructions stored therein.

While the present disclosure has been described with specific embodiments thereof, it is evident that many alternatives, modifications, and variations may be apparent to those skilled in the art. For example, various components of the embodiments may be interchanged, added, or substituted in the other embodiments. Also, all of the elements of each figure are not necessary for operation of the disclosed embodiments. For example, one of ordinary skill in the art of the disclosed embodiments would be able to make and use the teachings of the present disclosure by simply employing the elements of the independent claims. Accordingly, embodiments of the present disclosure as set forth herein are intended to be illustrative, not limiting. Various changes may be made without departing from the spirit and scope of the present disclosure.

Even though numerous characteristics and advantages of the present disclosure have been set forth in the foregoing description, together with details of the structure and function of the invention, the disclosure is illustrative only. Changes may be made to details, especially in matters of shape, size, and arrangement of parts, within the principles of the invention to the full extent indicated by the broad general meaning of the terms in which the appended claims are expressed.

What is claimed is:

1. A method of processing cytometric data, comprising:
dividing a first data matrix into a first plurality of first submatrices, the first data matrix indicative of a first plurality of properties of a first set of cells;
encoding each of the first submatrices into one corresponding vector representation to acquire a first plurality of vector representations; and
aggregating the first plurality of vector representations into a first ensemble representation.

2. The method of claim 1, further comprising:
dividing a second data matrix into a second plurality of second submatrices, the second data matrix indicative of a second plurality of properties of a second set of cells;
encoding each of the second submatrices into one corresponding vector representation to acquire a second plurality of vector representations;
aggregating the second plurality of vector representations into a second ensemble representation.

3. The method of claim 2, further comprising:
dividing a third data matrix into a third plurality of third submatrices, the third data matrix indicative of a third plurality of properties of a third set of cells;
encoding each of the third submatrices into one corresponding vector representation to acquire a third plurality of vector representations;
aggregating the third plurality of vector representations into a third ensemble representation;
concatenating the first, second, and third ensemble representations to acquiring a concatenated representation; and
classifying the cytometric data based on the concatenated representation.

4. The method of claim 1, wherein encoding each of the first submatrices includes transposing each of the first submatrices.

5. The method of claim 1, wherein each of the first submatrices is encoded into one corresponding vector representation based on at least one of: a Fisher Vector (FV) encoding method or a Vector of Locally Aggregated Descriptors (VLAD) encoding method.

6. The method of claim 5, further comprising:
applying a gradient updating function to at least one of: the FV encoding method or the VLAD encoding method,
wherein a NetFV encoding network is derived by applying the gradient updating function to the FV encoding method, and
wherein a NetVLAD encoding network is derived by applying the gradient updating function to the VLAD encoding method.

7. The method of claim 1, wherein aggregating the first plurality of vector representations into the first ensemble representation includes aggregating the first plurality of vector representations based on feature dimensions of the first plurality of vector representations.

8. The method of claim 7, wherein aggregating the first plurality of vector representations based on the feature dimensions includes performing at least one of following functions to each feature dimension of the first plurality of vector representations:
a majority voting function, a maximum pooling function, a mean pooling function, a stochastic pooling function, or a median pooling function.

9. The method of claim 7, wherein the number of the feature dimensions of the first plurality of vector representations is associated with the number of the first plurality of properties.

10. The method of claim 1, wherein the first data matrix is acquired from antibody-fluorescence measurements of the first set of cells.

11. A device of processing cytometric data, comprising:
a processor; and
a memory coupled with the processor,
wherein the processor executes computer-readable instructions stored in the memory to cause the device to perform operations, and the operations comprise:
receiving a first data matrix indicative of a first plurality of properties of a first set of cells;
by means of the processor, dividing the first data matrix into a first plurality of first submatrices;
by means of the processor, encoding each of the first submatrices into one corresponding vector representation to acquire a first plurality of vector representations; and
by means of the processor, aggregating the first plurality of vector representations into a first ensemble representation.

12. The device of claim 11, wherein the operations further comprise:
receiving a second data matrix indicative of a second plurality of properties of a second set of cells;
by means of the processor, dividing the second data matrix into a second plurality of second submatrices;
by means of the processor, encoding each of the second submatrices into one corresponding vector representation to acquire a second plurality of vector representations; and
by means of the processor, aggregating the second plurality of vector representations into a second ensemble representation.

13. The device of claim 12, wherein the operations further comprise:
receiving a third data matrix indicative of a third plurality of properties of a third set of cells;
by means of the processor, dividing the third data matrix into a third plurality of third submatrices;
by means of the processor, encoding each of the third submatrices into one corresponding vector representation to acquire a third plurality of vector representations;
by means of the processor, aggregating the third plurality of vector representations into a third ensemble representation;
by means of the processor, concatenating the first, second, and third ensemble representations to acquire a concatenated representation; and
by means of the processor, classifying the cytometric data based on the concatenated representation.

14. The device of claim 11, wherein encoding each of the first submatrices includes transposing each of the first submatrices.

15. The device of claim 11, wherein each of the first submatrices is encoded into one corresponding vector representation based on at least one of: a Fisher Vector (FV) encoding method or a Vector of Locally Aggregated Descriptors (VLAD) encoding method.

16. The device of claim 15, wherein the operations further comprise:
applying a gradient updating function to at least one of: the FV encoding method or the VLAD encoding method,
wherein a NetFV encoding network is derived by applying the gradient updating function to the FV encoding method, and
wherein a NetVLAD encoding network is derived by applying the gradient updating function to the VLAD encoding method.

17. The device of claim 11, wherein aggregating the first plurality of vector representations into the first ensemble representation includes aggregating the first plurality of vector representations based on feature dimensions of the first plurality of vector representations.

18. The device of claim 17, wherein aggregating the first plurality of vector representations based on the feature dimensions includes performing at least one of following functions to each feature dimension of the first plurality of vector representations:
a majority voting function, a maximum pooling function, a mean pooling function, a stochastic pooling function, or a median pooling function.

19. The device of claim 17, wherein the number of the feature dimensions of the first plurality of vector representations is associated with the number of the first plurality of properties.

20. The device of claim 11, wherein the first data matrix is acquired from antibody-fluorescence measurements of the first set of cells.
